(19) United States
(12) Patent Application Publication
(10) Pub. No.: US 2005/0209467 A1
(43) Pub. Date: Sep. 22, 2005

(54) Process for the preparation of 1 - (3-dimethylaminopropyl) -1 - (4-fluorophenyl) -1,3-dihydroisobenzofuran-5-carbonitrile

(75) Inventors: Leif Hilden, Kauniainen (FI); Petteri Rummakko, Espoo (FI); Arne Grumann, Kauniainen (FI); Pekka Pietikaeinen, Espoo (FI)

Correspondence Address: OBLON, SPIVAK, MCCLELLAND, MAIER & NEUSTADT, P.C., 1940 DUKE STREET, ALEXANDRIA, VA, 22314, US

(73) Assignee: Fermion Oy, Espoo (FI)

(21) Appl. No.: 11/045,087

(22) Filed: Jan. 31, 2005

Related U.S. Application Data
(63) Continuation of application No. PCT/FI03/00557, filed on Jul. 10, 2003.
(60) Provisional application No. 60/419,150, filed on Oct. 18, 2002.

(30) Foreign Application Priority Data
Jul. 30, 2002 (FI) FI 20021421

(51) Int. Cl. C07D 307/87

(52) U.S. Cl. 549/467

(57) ABSTRACT

The present invention is directed to novel processes for the preparation of citalopram comprising halogenation of a phthalide compound of formula II, 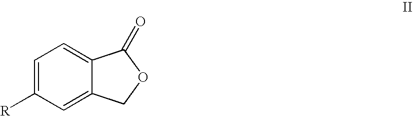
wherein R is a suitable group to be changed to CN, to afford an acid halogenide compound of formula III 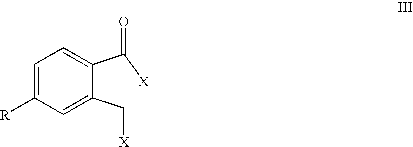 wherein R is as defined as above and X is halogen, and thereafter obtaining citalopram through two successive reactions with suitable organometallic halides or organoboranes or by a reaction with organometallic 4-fluorophenylhalide or 4-fluorophenylborane followed by reduction and alkylation, and an exchange of R to cyano to afford citalopram. The order of the reactions can be varied depending for example on the starting compound used.

### CROSS REFERENCES TO RELATED APPLICATIONS

The present application is a continuation application of International Patent Application No. PCT/FI2003/000557, filed on Jul. 10, 2003, and claims priority to Finnish Patent Application No. FI 20021421, filed on Jul. 30, 2002, and U.S. Provisional Application No. 60/419,150, filed on Oct. 18, 2002, all of which are incorporated herein by reference in their entireties.

The present invention relates to a novel method for the preparation of 1-(3-dimethylaminopropyl)-1-(4-fluorophenyl)-1,3-dihydroisobenzofuran-5-carbonitrile, which is a well known antidepressant, citalopram.

### BACKGROUND OF THE INVENTION

Citalopram is a selective, centrally acting serotonin (5-hydroxytryptamine; 5HT) reuptake inhibitor having antidepressant activity. This activity has been described e.g. in J. Hyttel, Prog. Neuro-Psychopharmacol. & Biol. Psychiat., 1982, 6, 277-295 and A. Gravem, Acta Psychiatr. Scand., 1987, 75, 478-486. In EP-A 474 580 it has been disclosed that citalopram has also effects in the treatment of dementia and cerebrovascular disorders.

Citalopram has the following structure:

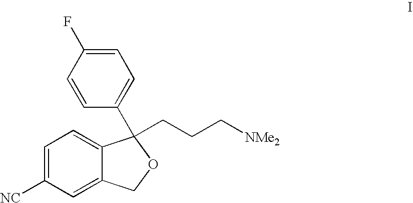

Citalopram was first described in DE 2,657,013 corresponding to U.S. Pat. No. 4,136,193. It was prepared by the reaction of 1-(4-fluorophenyl)-1,3-dihydro-5-isobenzofurancarbonitrile with a 3-(N,N-dimethylamino)propyl halide in the presence of a condensing agent. The starting material was prepared from the corresponding 5-bromo derivative by a reaction with cuprous cyanide. The other, in general terms outlined reaction comprises the ring closure of 5-bromo dihydroxy compound of formula A 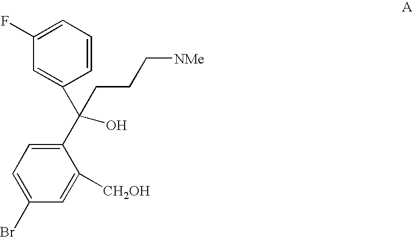
in the presence of a dehydrating agent. After the ring closure the 5-bromo group is replaced by a cyano group using cuprous cyanide. A compound of formula A is obtained from 5-bromophthalide by two successive Grignard reactions.

Other preparation methods are described e.g. in U.S. Pat. No. 4,650,884, U.S. Pat. No. 4,943,590, WO 98/19511, WO 98/19512, WO 98/19513, WO 99/30548, WO 2000/12044, WO 2000/13648 and WO 2000/23431.

In U.S. Pat. No. 4,943,590 preparation methods of individual enantiomers of citalopram are disclosed. In the process described dihydroxy compound of formula B 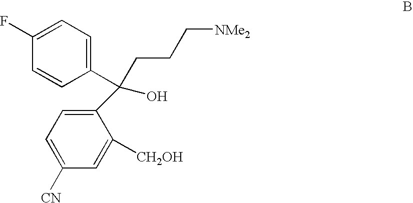
is first transferred into an ester and ring closure is then achieved in the presence of a base.

In WO 02/060886 there is discribed a process where 5-cyanophthalide is first halogented and thereafter citalopram is obtained via two Grignard reactions.

### SUMMARY OF TE INVENTION

The present invention is directed to novel processes for the preparation of citalopram comprising halogenation of a phthalide compound of formula II, 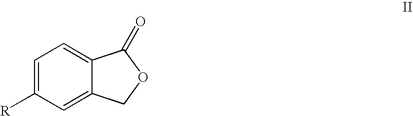
wherein R is a suitable group to be changed to CN, to afford an acid halogenide compound of formula III 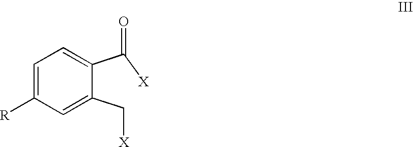
wherein R is as defined above and X is halogen, and threafter obtaining citalopram through two successive reactions with suitable organometallic halides or organoboranes or by a reaction with organometallic 4-fluorophenylhalide or 4-fluorophenylborane followed by reduction and alkylation, and an exchange of R to cyano to afford citalopram. The order of the reactions can be varied depending e.g. on the starting compound used. The benefit of the first step to the acid chloride is a higher selectivity of the following reaction.

Resulting citalopram can be purified by methods known in the art and it is isolated as the base or a pharmaceutically acceptable salt thereof.

Another aspect of the present invention are novel intermediate compounds of formula IIa and IVa 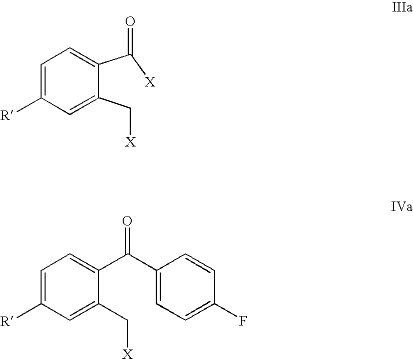
wherein R′ is as defined in claim 2 and X is a halogen excluding from compounds of formula ma those wherein R′ is CN, R¹ is Cl or Br while X is Br, or R′ is I while X is Cl, and from compounds of formula IVa those compounds where R′ is CN.

Still another aspect of the present invention are the preparation methods of novel intermediate compounds of formula IIIa and IVa.

Halogen means chloro, bromo, or iodo.

Alkyl means branched or unbranched alkyl groups having 1 to 6 carbon atoms, inclusive. Examples are methyl, ethyl, 1-propyl, 2-propyl, 1-butyl, 2-butyl, 2-methyl-2-propyl, etc.

Aryl means mono or bicyclic carboxylic aromatic group, such as phenyl and naphthyl, in particular phenyl or ring substituted phenyl. Also heteroaryl groups are possible.

Protective group R¹ in —CH₂OR¹ can be e.g. methyl, benzyl, allyl, or any other group which protects 0 from the reaction with halogenation agents used.

Protective groups R² and R³ in —NR²R³ can be e.g. alkyl or aryl, e.g. benzyl or allyl, or alkylcarbonyl, e.g. acetyl.

The process of the present invention from the selected cyanophthalide compounds to citalopram via acid halogenide is not described in any of the patents mentioned or in any other publication known.

### DETAILED DESCRIPTION OF THE INVENTION

Surprisingly, it has been found that if certain phthalide compounds used as starting materials in the citalopram process are halogenated, the reaction of the resulting compound of formula III or IIIa with an organometallic 4-fluorophenylhalide or with a 4-fluorophenyl borane is very selective and the following reactions give citalopram in good yield and purity. Citalopram can be prepared from compounds of formula III or IIIa via two Grignard reactions, using first organoborane reagents and thereafter Grignard reaction, or it can be prepared by Grignard or organoboron reaction followed by reduction of the ketone and ring closure and alkylation with the dimethylaminopropyl moiety. If 5-substituent is not cyano, the compound is cyanated in a suitable phase by methods known in the art.

First aspect of the present invention is a method for the preparation of 1-(3-dimethylaminopropyl)-1-(4-fluoro-phenyl)-1,3-dihydroisobenzofuran-5-carbonitrile of formula I 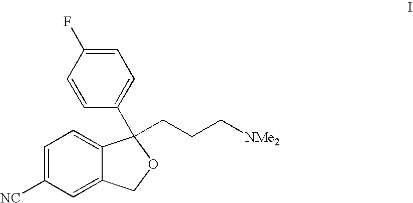
comprising the steps:
a) halogenation of a compound of formula II 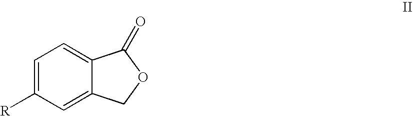
   wherein R is selected from the group consisting of:
      CH₂OR¹, wherein R¹ is a protective group,
      CH₃,
      COOR⁴, wherein R⁴ is alkyl or aryl,
      CONR⁵R⁶, wherein R⁵ is hydrogen and R⁶ is selected from hydrogen or alkyl or aryl, which can be substituted,
      C(OR⁷)₂, wherein R⁷ is alkyl
      oxazolinyl,
      thiazolinyl,
   or R is any other suitable group which can be converted to cyano,
   thereby obtaining a compound of formula III 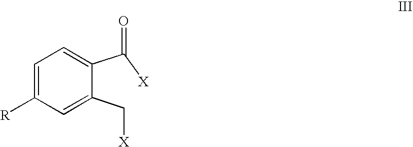
   wherein R is as defined above and X is halogen,
b) the reaction of a compound of formula III with an organometallic 4-fluorophenyl halide or 4-fluorophenylborane to afford a compound of formula IV 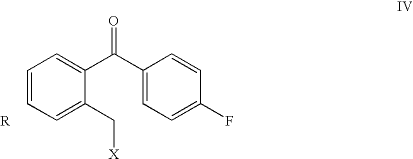
   wherein R and X are as defined above,
c) changing R to CN to afford a compound of formula V 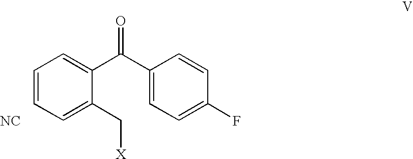
   and thereafter
d) either a reaction of a compound of formula V with organometallic dimethylaminopropyl halide to afford a compound of formula I; or a reduction of a compound of formula V to afford a compound of formula VI, 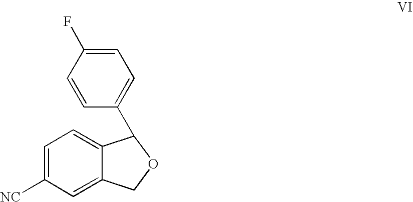
   which is alkylated to afford citalopram.

Another aspect of the present invention is a method for the preparation of 1-(3-dimethylaminopropyl)-1-(4-fluoro-phenyl)-1,3-dihydroisobenzofuran-5-carbonitrile comprising the steps:
a) halogenation of a compound of formula IIa, 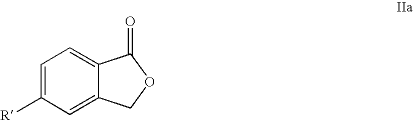
   wherein R′ is selected from the group consisting of:
      cyano,
      halogen,
      OSO₂(CF₂)ₙCF₃,
      CH₂OR¹, wherein R¹ is a protective group,
      NR²R³, wherein R² and R³ are independently either hydrogen or a protective group,
      CH₃,
      COOR⁴, wherein R⁴ is alkyl or aryl,
      CONR⁵R⁶, wherein R⁵ is hydrogen and R⁶ is selected from hydrogen, alkyl, or aryl,
      C(OR⁷)₂, wherein R⁷ is alkyl,
      oxazolinyl,
      thiazolinyl,
      OR⁸, wherein R⁸ is methyl, alkanoyl with 2 to 5 carbons, alkoxymethyl, 1-alkoxyethyl, benzyl or allyl,
   or R¹ is any other suitable group which can be converted to cyano,
   thereby obtaining a compound of formula IIIa, wherein R′ is as defined above and X is halogen,
b) the reaction of a compound of formula IIIa with an organometallic 4-fluorophenyl halide or 4-fluorophenylborane to afford a compound of formula IVa 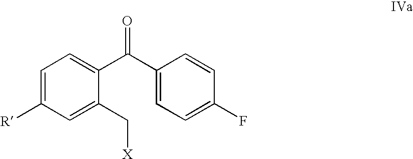
   wherein R′ and X are as defined above, and
c) a reduction of of a compound of formula IVa to afford a compound of formula VIIa, 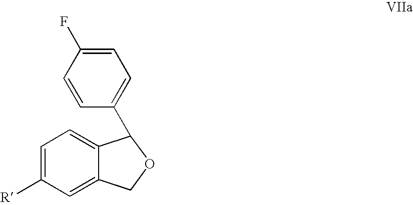
   which is then subjected to CN exchange and thereafter alkylated to afford citalopram.

Still another aspect of the present invention is a method for the preparation of 1-(3-dimethylaminopropyl)-1-(4-fluoro-phenyl)-1,3-dihydroisobenzofuran-5-carbonitrile comprising the steps:
a) halogenation of a compound of formula IIa, wherein R′ is selected from the group consisting of:
   cyano,
   halogen,
   OSO₂(CF₂)ₙCF₃,
   CH₂OR¹, wherein R¹ is a protective group,
   NR²R³, wherein R² and R³ are independently either hydrogen or a protective group,
   CH₃,
   COOR⁴, wherein R⁴ is alkyl or aryl,
   CONR⁵R⁶, wherein R⁵ is hydrogen and R⁶ is selected from hydrogen, alkyl, or aryl,
   C(OR⁷)₂, wherein R⁷ is alkyl,
   oxazolinyl,
   thiazolinyl,
   OR⁸, wherein R⁸ is methyl, alkanoyl with 2 to 5 carbons, alkoxymethyl, 1-alkoxyethyl, benzyl or allyl,
   or R is any other suitable group which can be converted to cyano,
   thereby obtaining a compound of formula IIIa, wherein R′ is as defined above and X is halogen,
b) the reaction of a compound of formula IIIa with an organometallic 4-fluorophenyl halide or 4-fluorophenylborane to afford a compound of formula IVa 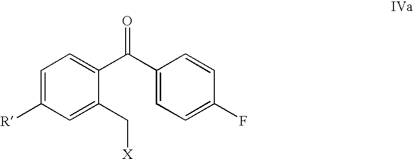
   wherein R′ and X are as defined above; thereafter
c) either a reaction of a compound of formula IVa with organometallic dimethylaminopropyl halide to afford a compound of formula VIIIa 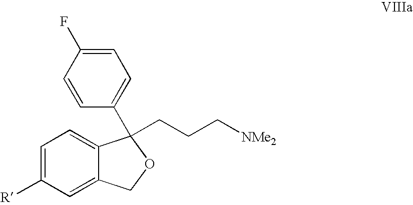
   which is then subjected to an exchange reaction R′ to CN, if needed, to afford citalopram; or a reduction of a compound of formula IVa to afford a compound of formula VIIa, 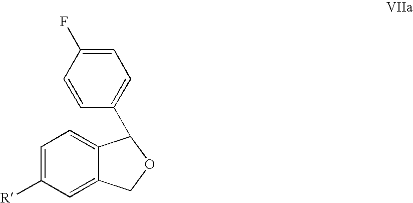
   which is alkylated and subjected to the exchange reaction R to CN, if needed, to afford citalopram.

Still another aspect of the present invention is a method for the preparation of a compound of formula VI, 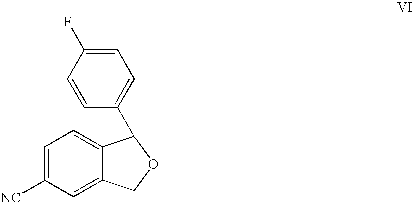
comprising reduction of a compound of formula IVa wherein X is a halogen and R′ is as defined in claim 2 excluding compounds where R′ is CN, and changing R′ to CN.

First step of the process is the halogenation of a compound of formula II or IIa to form the acid halogenide compound of formula III or IIIa where X is halogen, preferably chloro or bromo, most preferably chloro, and R is as defined in claim 1 and R′ as defined in claim 2.

The halogenation can be performed by any suitable method known in the art, eg. by the reaction with thionyl chloride in the presence of a suitable Lewis acid catalyst and a phase transfer catalyst. Catalysis by amines, e.g. pyridine or 4-dimethylaminopyridine or by N,N-dimethylformamide (DMF) is also possible. If DMF is used as a catalyst, no phase transfer catalyst is needed. Suitable Lewis acid catalysts are e.g. MgCl₂, MgBr₂, SnCl₂, SnCl₄, ZnCl₂, TiCl₄, AlCl₃, FeCl₃, BF₃Et₂O, BCl₃, B(OEt)₃, B(OMe)₃, B(O-iPr)₃, preferably boron based Lewis catalyst is used. Types of phase transfer catalyst used are halides of aromatic or aliphatic ammonium salts, for example tetramethylammonium chloride, tetrabutylammonium chloride or benzyl triethylammonium chloride. The catalyst is used 0.1 to 20 mol %, preferably 0.5 to 10 mol % based on the moles of phthalide derivative. The reaction with catalysts is preferably performed without any solvent, but if a solvent is used, any inert, high boiling solvent such as toluene, xylene, chlorobenzene or dichlorobenzene can be used.

The halogenation reagent used can be any suitable reagent used for halogenation, e.g. thionyl chloride, PCl₃, PCl₅, CCl₄ in triphenyl phosphine, oxalyl chloride or cyanuric chloride in trialkyl amine.

The reagents for preparing the corresponding bromo compound can be e.g. PBr₃, PBr₅, PPh₃Br₂, thionyl bromide or oxalyl bromide.

The halogenation reagent is used in the range from 0.5 to 1000 equivalents (based on phthalide derivative), preferably 1 to 10 equivalents, most preferably 1 to 5 equivalents. Reaction temperature can be from 20 to 150° C. or reflux temperature, preferably 80 to 140° C., most preferably 100 to 130° C. The reaction time is from 0.5 to 15 h, preferably less than 3 h.

The reaction will be completed readily and conversion is close to 100%. The product can be isolated and purified by suitable methods known in the art or the following step can be performed without purification of compound of formula m or IIIa.

Starting materials are either commercially available (e.g. if R′ is cyano or halogen) or they can be prepared by methods known in the art. If R′ is —OSO₂(CF₂)ₙCF₃ the compounds can be prepared from the corresponding hydroxy compounds by a conventional triflation reaction. —CH₂OR′ compounds can also be prepared from corresponding hydroxy compounds and —NR²R³ compounds from corresponding amino compounds by adding a suitable protective group by methods known in the art. Compounds with a CH₃-group can be prepared e.g. as described by Noguchi et al. in Heterocycles, 23(5), p. 1085-1088. Compounds where R or R′ is —COOR⁴ or —CONR₅R⁶ are commercialy available or they can be prepared from 5-carboxyphthalide by reaction with thionyl chloride and then with C₁₋₆ alkanol or C₁₋₆ alkylamine. If R or R′ is —C(OR⁷)₂ compounds can be prepared e.g. as described in WO 02/48133. Preparation of oxazolinyl and thiazolinyl compounds is described e.g. in WO 01/51477.

The advantage of making the acid halogenide is that the following reaction with an organometallic 4-fluorophenyl halide or with 4-fluorophenyl borane is very selective unlike the reaction of the lactone directly with 4-fluorophenylmagnesium halide, where the resulting ketone compound is more reactive than lactone and undesirable side products are formed.

The second step comprises the reaction of the acid halogenide compound of formula III or IIIa with an organometallic or organoboron reagent to afford the compound of formula IV or IVa.

The reagent used is a 4-fluorophenylborane or a 4-fluorophenylmetallo halide, wherein the metallic component can be Mg, Li, Cu, or Zn, preferably Mg or Cu. Preferably the reagent is a 4-fluorophenylmagnesium halide or a Grignard reagent of a 1-halide substituted 4-fluorobenzene, wherein the halogen component is preferably Cl or Br. Most preferably 4-fluorophenylmagnesium bromide is used. The amount of the reagent used is from 0.5 to 2.5 equivalents, preferably from 1 to 1.5 equivalents, based on the equivalents of the compound of formula III or IIIa.

The reaction is carried out in an inert organic solvent such as toluene or dimethylformamide or in commonly used ethers such as tetrahydrofuran, diethylether, di-n-butylether, tetrabutylmethyl ether, ethylene glycol dimethyl ether, 1,4-dioxane or mixtures thereof. The preferred solvents are tetrahydrofuran and ethylene glycol dimethyl ether or their mixtures with toluene. Cu, Ni, Pd, Ti, Fe or Zn compounds can be used as catalysts, preferably the reaction is performed without any catalyst. Reaction temperature is −80 to 60° C., preferably −20 to 20° C. The reaction is selective and the resulting compound of formula IV or IVa can be isolated and purified by crystallization or any other suitable method known in the art. The following reaction can also be performed without isolation of the intermediate of formula IV or IVa.

The following step can be the exchange of R or R′ group to cyano group, whereafter citalopram is achieved by the reaction of compound of formula V with an organometallic 3-dimethylaminopropylhalide with a spontaneous ring closure to citalopram. The other possibility is the reduction of the compound of formula V and thereafter alkylation to afford citalopram. Citalopram can be purified by methods known in the art and it can be isolated as a base or as a pharmaceutically suitable salt.

The metallo component of the organometallic 3-dimethylaminopropyl halide reagent used can be Mg, Li, Cu, or Zn, preferably Mg or Cu, most prefereably Mg. Preferably the reagent is a Grignard reagent of a 3-(N,N-dimethylamino)propyl halide, wherein the halide is Cl or Br. Most preferably the reagent is 3-(N,N-dimethylamino)propylmagnesiumchloride. The reaction is carried out in an inert organic solvent such as toluene or dimethylformamide or in commonly used ethers such as tetrahydrofuran, diethylether, di-n-butylether, tetrabutylmethyl ether, ethylene glycol dimethyl ether or 1,4-dioxane or mixtures thereof. The preferred solvents are tetrahydrofuran or ethylene glycol dimethyl ether or their mixtures with toluene. Cu, Ni, Pd, Ti, Fe or Zn compounds can be used as catalysts, preferably the reaction is performed without any catalyst. Reaction temperature is −80 to 60° C., preferably −20 to 20° C. and the reaction time is from 0.5 to 15 h, preferably less than 3 h. The organometallic reagent is used from 0.5 to 2.5 equivalents, preferably from 1 to 1.5 equivalents, based on the equivalents of the compound of formula IV or IVa.

The reduction can be performed by methods well known in the art and the alkylation can be performed e.g. as described in EP 1125907.

The exchange reaction R or R′ to cyano can be performed by methods known in the art. If R′ is halogen or —OSO₂(CF₂)ₙCF₃, the reaction with different cyanide compounds can be used e.g. as described in WO 00/13648 or in WO 00/11926.

Amide compounds can be converted to cyano compound e.g. as described in WO 01/66536 or in WO 99/30548 and amines as described in WO 98/19512.

—CH₃-group is first oxidized to an acid and thereafter reacted with a primary amine to an amide, which is transferred to cyano as above described.

If R or R′ is —C(OR⁷)₂, the conversion can be made first to aldehyde and then to cyano using standard procedures.

When R or R′ is an oxazoline or thiazoline, the conversion to cyano can be performed as described in WO 00/23431.

—OR-compounds are first deprotected to the corresponding alcohols which are sulphonated to a —OSO₂(CF₂)ₙCF₃— group.

Another route to citalopram comprises the reaction of a compound of formula IVa where R′ is as defined in claim 2 excluding compounds where R′ is CN, with an organometallic 3-dimethylaminopropyl halide to afford a compound of formula VIIIa, which is then subjected to the exchange reaction R′ to CN to afford citalopram.

Still one possibility is the reduction of the compound of formula IVa to afford a compound of formula VIIa, which is then subjected to the exchange reaction R¹ to cyano, if needed, and alkylated to citalopram, or first alkylated and thereafter R¹ is changed to cyano to afford citalopram. The alkylation can be performed e.g. as described in U.S. Pat. No. 4,136,193 or in EP 1 125 907.

All the reactions from phthalide to citalopram can be performed in one pot which makes the process convenient and saves costs and labour when no isolation or purification processes of intermediates are needed.

The compound of formula I may be used as a free base or as a pharmaceutically acceptable acid addition salt thereof. The acid addition salts can be prepared by methods known in the art.

The following examples merely illustrate the invention and they are not to be construed as limiting.

### EXAMPLE 1

2-Chloromethyl-4-chloro-benzoyl chloride
1-Oxo-5-chloro-1,3-dihydro-isobenzofuran (20 g), boron trifluoride etherate (0.8 ml), benzyl triethyl ammonium chloride (2,2 g) thionyl chloride (13 ml) and xylene (100 ml) were suspended into reaction vessel. The mixture was heated to reflux for 20 hours (temp 120-130° C.). Excess of thionyl chloride and the solvent were distilled off under reduced pressure. The crude product was purified by distillation under reduced pressure using kugelrohr aparatus. Yield: 10,2 g, 39% viscous oil. ¹H NMR (CDCl₃, 400 MHz): 4.7δ (2H, s), 7.40 (1H, dd, J=1, 8 Hz), 7.57 (1H, d, J=1 Hz), 8.13 (1H, d, J=8 Hz).
1-(4-fluorophenyl)-1-(3-dimethyl aminopropyl)-5-chlorophthalane

A solution of 4-fluoro phenylmagnesium bromide 1M in tetrahydrofuran (25 ml) was added to a cooled solution of 2-chloromethyl-4-chloro-benzoyl chloride 2 (5.0 g) in toluene (35 ml) so that the temperature did not raise above 0° C. The mixture was stirred for two hours in 0° C. After 2 hours reaction time freshly prepared 3-dimethylaminopropylmagnesium chloride (0,79 mol/kg 21,5 ml) was added slowly to cooled reaction mixture at 0° C. The reaction was quenched after 2 hours by addition of water (50 ml) and the pH of the mixture was adjusted to 4-4,5 by addition of concentrated acetic acid. After phase separation the pH of the water layer was adjusted to 8-8,5 by addition of ammonia (25%). Extraction with toluene and evaporation yielded crude product (3,8 g) as brown oil. ¹H NMR (CDCl3, 400 MHz): 1.39 (1H, m), 1.52 (1H, m), 2.16-2.31 (4H, m), 2,19 (6H, s), 5,16 (dd, J=1, 8 Hz), 7,02-7,50 (7H, m).
1-(3-dimethylaminopropyl)-1-(4-fluorophenyl)-1,3-dihydroisobenzofuran-5-carbonitrile

Triphenylphosphine (3.1 g, 0.012 mol) and nickel dichloride (0,38 g, 0.003 mol) in acetonitrile (120 ml) were refluxed for one hour. Zinc powder (0.2 g, 0.003 mol) and a solution of 1-(4-fluorophenyl)-1-(3-dimethylaminopropyl)-5-chlorophthalane (5 g) in warm acetonitrile (100 ml) were added with a time delay of 10 minutes. The mixture was cooled to room temperature and sodium cyanide (1.5 g, 0.03 mol) was added. After refluxing for 15 hours the mixture was cooled to room temperature and filtered. The filtrate was concentrated under reduced pressure followed by refluxing of the residue in water (200 ml) for 20 minutes. Filtration and drying in vacuo gave the crude product as an oil.

### EXAMPLE 2

1-(4-fluorophenyl)-1,3-dihydroisobenzofuran-5-carbonitrile

To a suspension of 3-chloromethyl-4-(4-fluoro benzoyl)benzonitrile (1,0 g) in ethanol (10 ml) was added solid NaBH₄ (0,07 g). The mixture was allowed to stir in room temperature overnight. The reaction was quenched by addition on water (5 ml) and the mixture was stirred for two hours. The white precipitate (0,18 g) was filttered and analysed by NMR and GC-MS. The yield was 21%. ¹H NMR (CDCl₃, 400 MHz): 5,12 (1H, d, J=12, 5 Hz), 5,26 (1H, d, J=12,5 Hz),6,08 (1H, s), 6,95-7,60 (7H).
1-(3-dimethylaminopropyl)-1-(4-fluorophenyl)-1,3-dihydroisobenzofuran-5-carbonitrile

Diisopropylamine (1 g, 0.01 mol) was added to tetrahydrofuran (5 ml) and cooled to +20° C. Butyl lithium (5.7 ml, 0.009 mol) was added dropwise and the mixture was stirred for 1 hour while the mixture warmed up to room temperature. After re-cooling to −20° C. a solution of 1-(4-fluorophenyl)-1,3-dihydroisobenzofuran-5-carbonitrile (2 g, 0.008 mol) in tetrahydrofuran (15 ml) was added dropwise, the mixture was stirred for 30 minutes and a solution of 3-(dimethylamino) propyl chloride (1.22 g, 0.01 mol) in tetrahydrofuran (5 ml) was added slowly. The reaction mixture was warmed up over night and water (30 ml) was added. Extraction of the aqueous phase with toluene, drying over magnesium sulfate, filtration and evaporation to dryness gave crude citalopram.

Obviously, numerous modifications and variations of the present invention are possible in light of the above teachings. It is therefore to be understood that, within the scope of the appended claims, the invention may be practiced otherwise than as specifically described herein.

1: A method for the preparation of 1-(3-dimethylaminopropyl)-1-(4-fluoro-phenyl)-1,3-dihydroisobenzofuran-5-carbonitrile of formula I 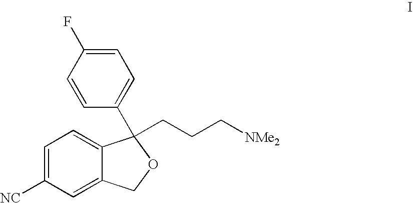
comprising:
a) halogenating a compound of formula II, 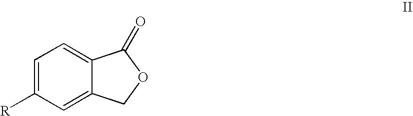
wherein R is selected from the group consisting of
CH₂OR¹, wherein R¹ is a protective group,
CH₃,
COOR⁴, wherein R⁴ is alkyl or aryl
CONR⁵R⁶, wherein R⁵ is hydrogen and R⁶ is hydrogen, alkyl, or aryl,
C(OR⁷)₂, wherein R⁷ is alkyl
oxazolinyl,
thiazolinyl, and
any other suitable group which can be converted to cyano,
to obtain a compound of formula III 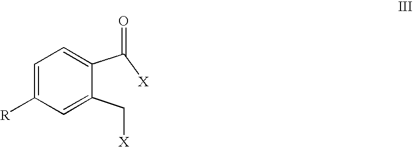
wherein X is halogen,
b) reacting said compound of formula III with an organometallic 4-fluorophenyl halide or 4-fluorophenylborane to obtain a compound of formula IV 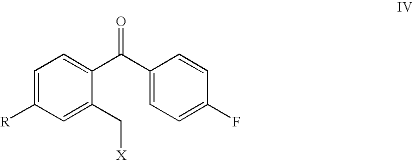
c) changing R to CN to obtain a compound of formula V 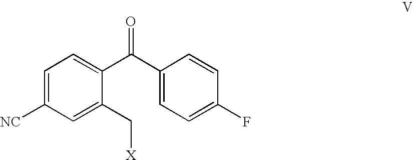
and then
d) reacting said compound of formula V with organometallic dimethylaminopropyl halide to obtain said compound of formula I; or reducing said compound of formula V to obtain a compound of formula VI, 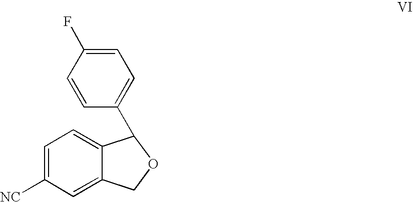
which is alkylated to obtain said 1-(3-dimethylaminopropyl)-1-(4-fluoro-phenyl)-1,3-dihydroisobenzofuran-5-carbonitrile.

2: The method of claim 1 wherein the reaction of step d) is the reduction of a compound of formula V.

3: The method of claim 1, wherein said compound of formula III is reached with 4-fluorophenyl magnesium bromide.

4: A method for the preparation of 1-(3-dimethylaminopropyl)-1-(4-fluoro-phenyl)-1,3-dihydroisobenzofuran-5-carbonitrile comprising:
a) halogenating a compound of formula IIa, 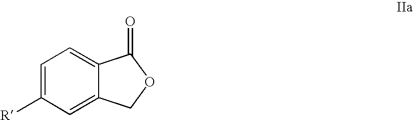
wherein R′ is selected from the group consisting of
cyano,
halogen,
OSO₂(CF₂)ₙCF₃,
CH₂OR¹, wherein R¹ is a protective group,
NR²R³, wherein R² and R³ are independently either hydrogen or a protective group,
CH₃,
COOR⁴, wherein R⁴ is alkyl or aryl,
CONR⁵R⁶, wherein R⁵ is hydrogen and R⁶ is hydrogen, alkyl, or aryl,
C(OR⁷)₂, wherein R⁷ is alkyl,
oxazolinyl,
thiazolinyl,
OR⁸, wherein R⁸ is methyl, alkanoyl with 2 to 5 carbons,
alkoxymethyl, 1-alkoxyethyl, benzyl or allyl, and
any other suitable group which can be converted to cyano, to obtain a compound of formula IIIa, 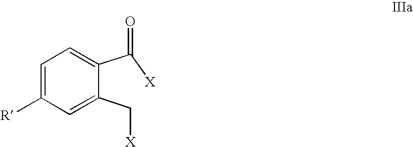
wherein X is halogen,
b) reacting said compound of formula IIIa with an organometallic 4-fluorophenyl halide or 4-fluorophenylborane to obtain a compound of formula IVa, 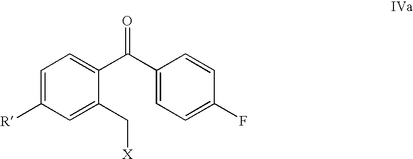
and
c) reducing said compound of formula IVa to obtain a compound of formula VIIa, 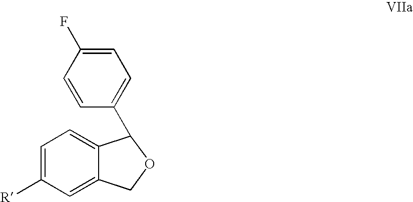
and, if R′ is different from CN, said compound of formula VIIa is subjected to CN exchange to afford compound of formula VI 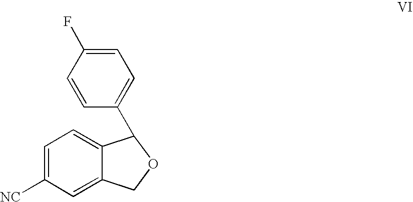
which is thereafter alkylated to obtain said 1-(3-dimethylaminopropyl)-1-(4-fluoro-phenyl)-1,3-dihydroisobenzofuran-5-carbonitrile.

5: A method for the preparation of 1-(3-dilnethylaminopropyl)-1-(4-fluoro-phenyl)-1,3-dihydroisobenzofuran-5-carbonitrile comprising:
a) halogenating a compound of formula Ia, 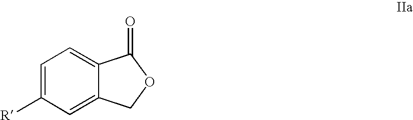
wherein R′ is selected from the group consisting of
cyano,
halogen,
OSO₂(CF₂)ₙCF₃,
CH₂OR¹, wherein R¹ is a protective group,
NR²R³, wherein R² and R³ are independently either hydrogen or a protective group,
CH₃,
COOR⁴, wherein R⁴ is alkyl or aryl,
CONR⁵R⁶, wherein R⁵ is hydrogen and R⁶ is hydrogen, alkyl, or aryl,
C(OR⁷)₂, wherein R⁷ is alkyl,
oxazolinyl,
thiazolinyl,
OR⁸, wherein R⁸ is methyl, alkanoyl with 2 to 5 carbons, alkoxymethyl, 1-alkoxyethyl, benzyl or allyl, and
any other suitable group which can be converted to cyano, to obtain a compound of formula IIIa, 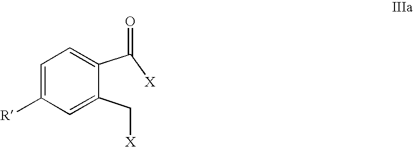
wherein X is a halogen,
b) reacting said compound of formula IIIa with an organometallic 4-fluorophenyl halide or 4-fluorophenylborane to obtain a compound of formula IVa, 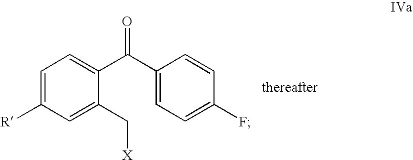
c) reacting said compound of formula IVa with organometallic dimethylanlinopropyl halide to obtain a compound of formula VIIIa 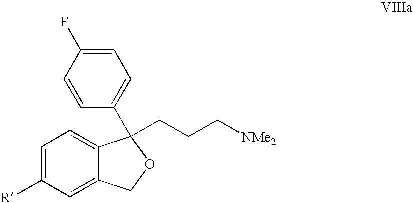
which is then subjected to an exchange reaction R′ to CN, if needed, to obtain said 1-(3-dimethylaminopropyl)-1-(4-fluoro-phenyl)-1,3-dihydroisobenzofuran-5-carbonitrile; or reducing said compound of formula IVa to obtain a compound of formula VIIa, 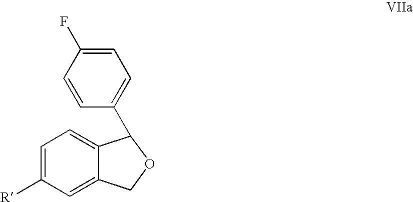
which is alkylated and subjected to the exchange reaction R′ to CN, if needed, to obtain 1-(3-dimethylaminopropyl)-1-(4-fluoro-phenyl)-1,3-dihydroisobenzofuran-5-carbonitrile.

6: The method of claim 5 wherein in step c) the compound of formula IVa is first reduced to obtain a compound of formula VIIa which is then alkylated and CN is introduced to obtain said 1-(3-dimethylaminopropyl)-1-(4-fluoro-phenyl)-1,3-dihydroisobenzofuran-5-carbonitrile.

7: A method for the preparation of the compound of formula I 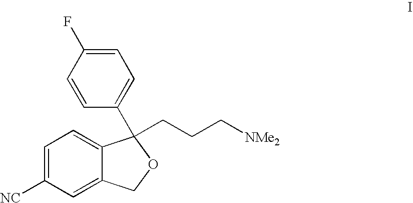
comprising reacting a compound of formula IV 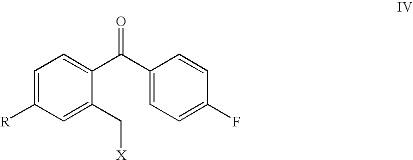
wherein X is a halogen and R is selected from the group consisting of
CH₂OR¹, wherein R¹ is a protective group,
CH₃,
COOR⁴, wherein R⁴ is alkyl or aryl
CONR⁵R⁶, wherein R⁵ is hydrogen and R⁶ is hydrogen, alkyl, or aryl,
C(OR⁷)₂, wherein R⁷ is alkyl
oxazolinyl, and
thiazolinyl, and
any other suitable group which can be converted to cyano, with an organometallic 3-dimethylaminopropyl halide and thereafter changing R to CN.

8: The method of claim 7, wherein the organometallic 3-dimethylaminopropyl halide is 3-(N,N-dimethylamino)propylmagnesium chloride.

9: A compound having formula IIIa 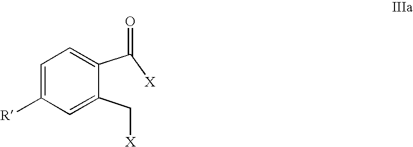
wherein X is halogen and R′ is selected from the group consisting of
cyano,
halogen,
OSO₂(CF₂)ₙCF₃,
CH₂OR¹, wherein R¹ is a protective group,
NR²R³, wherein R² and R³ are independently either hydrogen or a protective group,
CH₃,
COOR⁴, wherein R⁴ is alkyl or aryl,
CONR⁵R⁶, wherein R⁵ is hydrogen and R⁶ is hydrogen, alkyl, or aryl,
C(OR⁷)₂, wherein R⁷ is alkyl,
oxazolinyl,
thiazolinyl,
OR⁸, wherein R⁸ is methyl, alkanoyl with 2 to 5 carbons, alkoxymethyl, 1-alkoxyethyl, benzyl or allyl, and
any other suitable group which can be converted to cyano,
excluding compounds where R′ is CN; R′ is Cl or Br and X is Br; R′ is F and X is Cl; and R′ is I and X is Cl.

10: A method for the preparation of a compound of formula IIIa 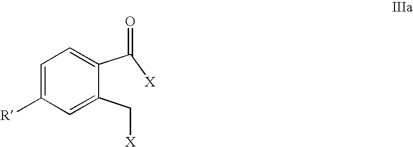
comprising halogenating a compound of formula IIa, 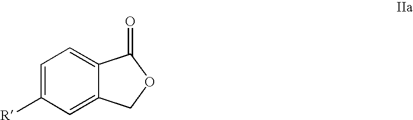
wherein X is a halogen and wherein R¹ is selected from the group consisting of
cyano,
halogen,
OSO₂(CF₂)ₙCF₃,
CH₂OR¹, wherein R¹ is a protective group,
NR²R³, wherein R² and R³ are independently either hydrogen or a protective group,
CH₃,
COOR⁴, wherein R⁴ is alkyl or aryl,
CONR⁵R⁶, wherein R⁵ is hydrogen and R⁶ is hydrogen, alkyl, or aryl,
C(OR⁷)₂, wherein R⁷ is alkyl,
oxazolinyl,
thiazolinyl,
OR⁸, wherein R⁸ is methyl, alkanoyl with 2 to 5 carbons, alkoxymethyl, 1-alkoxyethyl, benzyl or allyl, and
any other suitable group which can be converted to cyano.

11: A compound having formula IVa 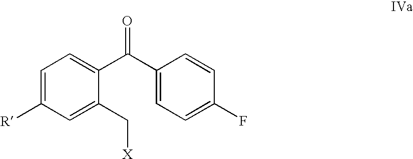
wherein X is halogen and wherein R′ is selected from the group consisting of
cyano,
halogen,
OSO₂(CF₂)ₙCF₃,
CH₂OR¹, wherein R¹ is a protective group,
NR²R³, wherein R² and R³ are independently either hydrogen or a protective group,
CH₃,
COOR⁴, wherein R⁴ is alkyl or aryl,
CONR⁵R⁶, wherein R⁵ is hydrogen and R⁶ is hydrogen, alkyl, or aryl,
C(OR⁷)₂, wherein R⁷ is alkyl,
oxazolinyl,
thiazolinyl,
OR⁸, wherein R⁸ is methyl, alkanoyl with 2 to 5 carbons, alkoxymethyl, 1-alkoxyethyl, benzyl or allyl, and
any other suitable group which can be converted to cyano, excluding compounds where R′ is CN, halogen, or CH₃.

12: A method for the preparation of a compound of formula IVa 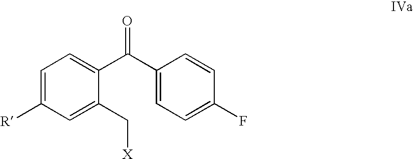
comprising reacting a compound of formula IIIa 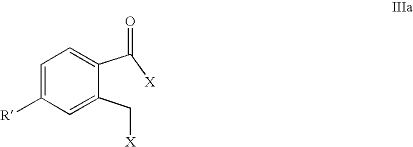
with a 4-fluorophenylborane or with 4-fluorophenylmetallo halide wherein R′ is selected from the group consisting of
cyano,
halogen,
OSO₂(CF₂)ₙCF₃,
CH₂OR¹, wherein R¹ is a protective group,
NR²R³, wherein R² and R³ are independently either hydrogen or a protective group,
CH₃,
COOR⁴, wherein R⁴ is alkyl or aryl,
CONR⁵R⁶, wherein R⁵ is hydrogen and R⁶ is hydrogen, alkyl, or aryl,
C(OR⁷)₂, wherein R⁷ is alkyl,
oxazolinyl,
thiazolinyl,
OR⁸, wherein R⁸ is methyl, alkanoyl with 2 to 5 carbons, alkoxymethyl, 1-alkoxyethyl, benzyl or allyl, and
any other suitable group which can be converted to cyano, excluding compounds where R′ in CN.

13: A method for the preparation of a compound of formula VI 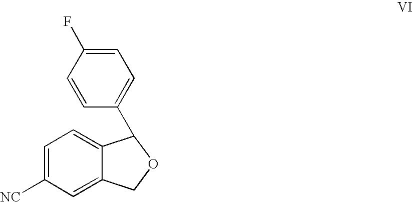
comprising reducing a compound of formula IVa 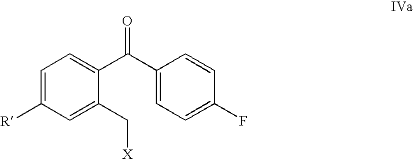
wherein X is a halogen and wherein R′ is selected from the group consisting of
cyano,
halogen,
OSO₂(CF₂)ₙCF₃,
CH₂OR¹, wherein R¹ is a protective group,
NR²R³, wherein R² and R³ are independently either hydrogen or a protective group,
CH₃,
COOR⁴, wherein R⁴ is alkyl or aryl,
CONR⁵R⁶, wherein R⁵ is hydrogen and R⁶ is hydrogen, alkyl, or aryl,
C(OR⁷)₂, wherein R⁷ is alkyl,
oxazolinyl,
thiazolinyl,
OR⁸, wherein R⁸ is methyl, alkanoyl with 2 to 5 carbons, alkoxymethyl, 1-alkoxyethyl, benzyl or allyl, and
any other suitable group which can be converted to cyano, excluding compounds where R′ is CN, and changing R′ to CN.
